SEQUENCE LISTING (42 sequences)

SEQ ID NO: 1 - protein, 119 aa, artificial sequence
  antibody sequence derived from library or synthesized
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSS

SEQ ID NO: 2 - protein, 111 aa, artificial sequence
  antibody sequence derived from library or synthesized
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGSAAA

SEQ ID NO: 3 - protein, 119 aa, artificial sequence
  antibody sequence derived from library or synthesized
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 4 - protein, 106 aa, artificial sequence
  antibody sequence derived from library or synthesized
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 5 - protein, 122 aa, artificial sequence [sequence omitted]

SEQ ID NO: 6 - protein, 113 aa, artificial sequence
  antibody sequence derived from library or synthesized
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTIKRT

SEQ ID NO: 7 - protein, 119 aa, artificial sequence; identical to SEQ ID NO: 3
  antibody sequence derived from library or synthesized

SEQ ID NO: 8 - protein, 106 aa, artificial sequence; identical to SEQ ID NO: 4
  antibody sequence derived from library or synthesized

SEQ ID NO: 9 - protein, 119 aa, artificial sequence
  antibody sequence derived from library or synthesized
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSAKT

SEQ ID NO: 10 - protein, 110 aa, artificial sequence
  antibody sequence derived from library or synthesized
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKRAD

SEQ ID NO: 11 - protein, 114 aa, artificial sequence
  antibody sequence derived from library or synthesized
QVQLQQSGPEDVKPGASVKISCKASGYTFTDYYMNWVKQSPGKGLEWIGDINPNNGGTSYNQKFKGRATLTVDKSSSTAYMELRSLTSEDSSVYYCESQSGAYWGQGTTVTVSA

SEQ ID NO: 12 - protein, 116 aa, artificial sequence
  antibody sequence derived from library or synthesized
DYKDILMTQTPSSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKR

SEQ ID NO: 13 - protein, 128 aa, artificial sequence
  antibody sequence derived from library or synthesized
EVQLVESGGGLVKPGGSLKLSCAVSGFTFSDYAMSWIRQTPENRLEWVASINIGATYAYYPDSVKGRFTISRDNAKNTLFLQMSSLGSEDTAMYYCARPGSPYEYDKAYYSMAYWGPGTSVTVSSAKT

SEQ ID NO: 14 - protein, 118 aa, artificial sequence
  antibody sequence derived from library or synthesized
DVQMTQSTSSLSASLGDRVTISCRASQDIKNYLNWYQQKPGGTVKLLIYYSSTLLSGVPSRFSGRGSGTDFSLTITNLEREDIATYFCQQSITLPPTFGGGTKLEIKRADAAPTVSIF

SEQ ID NO: 15 - protein, 10 aa, artificial sequence
  antibody sequence derived from library or synthesized
GYTFTRYTMH

SEQ ID NO: 16 - protein, 17 aa, artificial sequence
  antibody sequence derived from library or synthesized
YINPSRGYTNYADSVKG

SEQ ID NO: 17 - protein, 10 aa, artificial sequence
  antibody sequence derived from library or synthesized
YYDDHYCLDY

SEQ ID NO: 18 - protein, 10 aa, artificial sequence
  antibody sequence derived from library or synthesized
RASQSVSYMN

SEQ ID NO: 19 - protein, 7 aa, artificial sequence
  antibody sequence derived from library or synthesized
DTSKVAS

SEQ ID NO: 20 - protein, 9 aa, artificial sequence
  antibody sequence derived from library or synthesized
QQWSSNPLT

SEQ ID NO: 21 - protein, 10 aa, artificial sequence
  antibody sequence derived from library or synthesized
GYTFTRYTMH

SEQ ID NO: 22 - protein, 17 aa, artificial sequence
  antibody sequence derived from library or synthesized
YINPSRGYTNYNQKFKD

SEQ ID NO: 23 - protein, 10 aa, artificial sequence
  antibody sequence derived from library or synthesized
YYDDHYCLDY

SEQ ID NO: 24 - protein, 10 aa, artificial sequence
  antibody sequence derived from library or synthesized
RASSSVSYMN

SEQ ID NO: 25 - protein, 7 aa, artificial sequence
  antibody sequence derived from library or synthesized
DTSKVAS

SEQ ID NO: 26 - protein, 9 aa, artificial sequence
  antibody sequence derived from library or synthesized
QQWSSNPLT

SEQ ID NO: 27 - protein, 10 aa, artificial sequence
  antibody sequence derived from library or synthesized
GYSFTGYTMN

SEQ ID NO: 28 - protein, 17 aa, artificial sequence
  antibody sequence derived from library or synthesized
LINPYKGVSTYNQKFKD

SEQ ID NO: 29 - protein, 11 aa, artificial sequence
  antibody sequence derived from library or synthesized
YYGDSDWYFDV

SEQ ID NO: 30 - protein, 11 aa, artificial sequence
  antibody sequence derived from library or synthesized
RASQDIRNYLN

SEQ ID NO: 31 - protein, 7 aa, artificial sequence
  antibody sequence derived from library or synthesized
YTSRLES

SEQ ID NO: 32 - protein, 9 aa, artificial sequence
  antibody sequence derived from library or synthesized
QQGNTLPWT

SEQ ID NO: 33 - protein, 8 aa, artificial sequence
  antibody sequence derived from library or synthesized
GYTFTRYT

SEQ ID NO: 34 - protein, 8 aa, artificial sequence
  antibody sequence derived from library or synthesized
INPSRGYT

SEQ ID NO: 35 - protein, 12 aa, artificial sequence
  antibody sequence derived from library or synthesized
ARYYDDHYCLDY

SEQ ID NO: 36 - protein, 5 aa, artificial sequence
  antibody sequence derived from library or synthesized
SSVSY

SEQ ID NO: 37 - protein, 9 aa, artificial sequence
  antibody sequence derived from library or synthesized
QQWSSNPLT

SEQ ID NO: 38 - protein, 8 aa, artificial sequence
  antibody sequence derived from library or synthesized
GFTFSGYG

SEQ ID NO: 39 - protein, 8 aa, artificial sequence
  antibody sequence derived from library or synthesized
ITSSSINI

SEQ ID NO: 40 - protein, 9 aa, artificial sequence
  antibody sequence derived from library or synthesized
ARFDWDKNY

SEQ ID NO: 41 - protein, 6 aa, artificial sequence
  antibody sequence derived from library or synthesized
QDISNY

SEQ ID NO: 42 - protein, 9 aa, artificial sequence
  antibody sequence derived from library or synthesized
QQYYNYPWT

(19) United States
(12) Patent Application Publication
(10) Pub. No.: US 2021/0070846 A1
(43) Pub. Date: Mar. 11, 2021

(54) SPECIFIC DOSAGE REGIMEN FOR HEMIBODY THERAPY

(71) Applicant: Gernot Stuhler, Tübingen (DE)

(72) Inventor: Gernot Stuhler, Tübingen (DE)

(21) Appl. No.: 16/955,902

(22) PCT Filed: Dec. 21, 2018
(86) PCT No.: PCT/EP2018/086629

§ 371 Date: Jun. 19, 2020

(30) Foreign Application Priority Data
Dec. 21, 2017 (EP) 17209418.7

(51) Int. Cl. C07K 16/18

(52) U.S. Cl. CPC C07K 16/18; A61K 45/06; C07K 2317/76

(57) ABSTRACT

The present invention relates to a composition comprising at least two complimentary hemibodies, a kit comprising at least two compositions each comprising at least one hemibody, a dosage scheme of at least two pharmaceutical compositions, comprising hemibodies, and uses thereof.

### FIELD OF THE INVENTION

The present application relates to specific dosage regimen for hemibody therapy.

### BACKGROUND

The concept of hemibody therapy is broadly disclosed in WO2013104804, the content of which is incorporated herein by reference.

In short, hemibody therapy consists of two or more molecules, called hemibodies, each of which having a different targeting moiety, which binds to a different cell surface antigen, and a different fragment of a functional domain.

In case the two or more molecules bind to a cell which expresses the different cell surface antigens in sufficient surface density, the different fragments of the functional domain can associate or pair, to render the functional domain functional.

In such way, a tissue or cell which is characterized by expression of the two or more cell surface antigens can be labeled (in case the completed functional domain is a detectable label, like a fluorophore, an isotope, an enzyme or the like), or a therapeutic effect can be evoked (in case the completed functional domain is, e.g., a domain that engages or attracts T lymphocytes or other effector cells, or has a cytotoxic or cytostatic effect on its own).

The set of two or more hemibodies wherein the individual members have different fragments of the same functional domain, so that association or pairing thereof renders the functional domain functional, is called a complimentary set, or pair, herein.

One basic concept of hemibody therapy is to choose a surface antigen combination which is highly specific for a given pathological condition. Because the hemibodies only become efficacious when the complementary fractions of the functional domain have paired, binding on healthy tissue, where only one of the two or more surface antigens is expressed, does not evoke any effects, and therefore does not have side effects on healthy tissue.

By this means, a highly disease specific therapy can be established and the side effects can be reduced or even completely ruled out.

However, oftentimes there is no such typical surface antigen combination in particular pathological conditions.

Oftentimes, a given pathological condition is not characterized by whether a specific surface antigen is expressed or not (yes/no answer), but rather by the degree of expression of the specific surface antigen.

This means, for example, that a surface antigen which is considered to be a disease marker can be lowly expressed on healthy tissues, but highly expressed on pathological tissues.

Hence, a respective tissue can be characterized by normal or near-normal expression of a cell surface antigen A_{HK} which is expressed under normal and pathological conditions (“housekeeper”), and by overexpression of a cell surface antigen A_{DM} which is indicative for a given pathological condition (“disease marker”).

The inventors have surprisingly found that differential dosing of two or more complimentary hemibodies can help to increase the specificity and thus the field of therapeutic applications for this new type of therapeutic products. On the other hand, off target effects can thus be reduced.

For example in the case of breast cancer, Her2/neu is overexpressed in about 15-30% of patients. Hence, Her2/neu is a major target of antibody therapy, e.g., with the therapeutic antibody Herceptin.

In contrast thereto, EpCAM is present at the basolateral surface of virtually all simple epithelia but not on muscle cells, including heart muscle cells. Of note, Her2/neu is expressed on heart muscle cells and one of the most prominent on-target/off-tumor effects of Herceptin treatment is heart damage.

In some immunotherapies, like bispecific antibody triggered T cell engagement or CAR T cell therapy, only few targeted surface antigens per cell suffice to attract T lymphocytes and to evoke a cytotoxic or cytolytic response. Hence, surface antigens which are expressed both in healthy and pathological tissue, irrespective of their density (above a certain threshold or density), are not optimal targets for such therapy. Indeed, a CAR against Her2/neu elicited early death in one patient treated (Morgan et al., 2010).

Hence, while Her-2/neu is usually perceived to be a disease marker, the mere fact that it is also expressed in healthy tissue, though only in small copy numbers, makes it unsuitable for such types of therapy.

Likewise, high toxicity was observed for bispecific antibodies directed against EpCAM.

Now there is coexpression of Her2/neu and EpCAM in non-pathologic tissues and cells, like endocrine tissues, nasopharaynx and bronchus, some gastrointestinal tract tissues and other cells.

For this reason, the mere fact that these two surface antigens are expressed on the same cells does not suffice as a criterion for hemibody therapy. The mere combination alone is oftentimes simply not specific enough to be used as a selection criterion.

It is hence one object of the present invention to increase the specificity and reduce off-tumor effects for hemibody therapy.

It is hence one further object of the present invention to make hemibody therapy usable in diseases which do not have a tumor-exclusive marker profile where the presence of two or more markers characterizes a cancer cells and unambiguously distinguishes it from healthy tissue.

These and further objects are met with methods and means according to the independent claims of the present invention. The dependent claims are related to specific embodiments.

### SUMMARY OF THE INVENTION

The present invention provides a composition comprising at least two complimentary hemibodies with specifically adapted dosages. The invention further provides a kit comprising at least two compositions each comprising at least one hemibody each, with specifically adapted dosages. The invention further provides a dosage scheme of at least two pharmaceutical compositions comprising at least one hemibody each, with specifically adapted dosages.

The invention and general advantages of its features will be discussed in detail below.

### DESCRIPTION OF THE FIGURES

The following abbreviations are used: Construct 55=hemibody against CD45 (antiCD3VH-antiCD45scFv). Construct 42=hemibody against HLA-A2 (antiCD3VL-antiHLA-A2scFv). BiTE=antiCD3scFv-antiHLA-A2scFv (BiTE=Bispecifc T-Cell engager).

FIG. 1 shows a situation where the dosage ratio of the complimentary hemibodies has been adapted according to the surface density of the two respective antigens in a pathological tissue. In said tissue, a disease marker DM (antigen 1) is overexpressed, while a housekeeper HK (antigen 2) is expressed at physiological levels in transformed and non-transformed tissues.

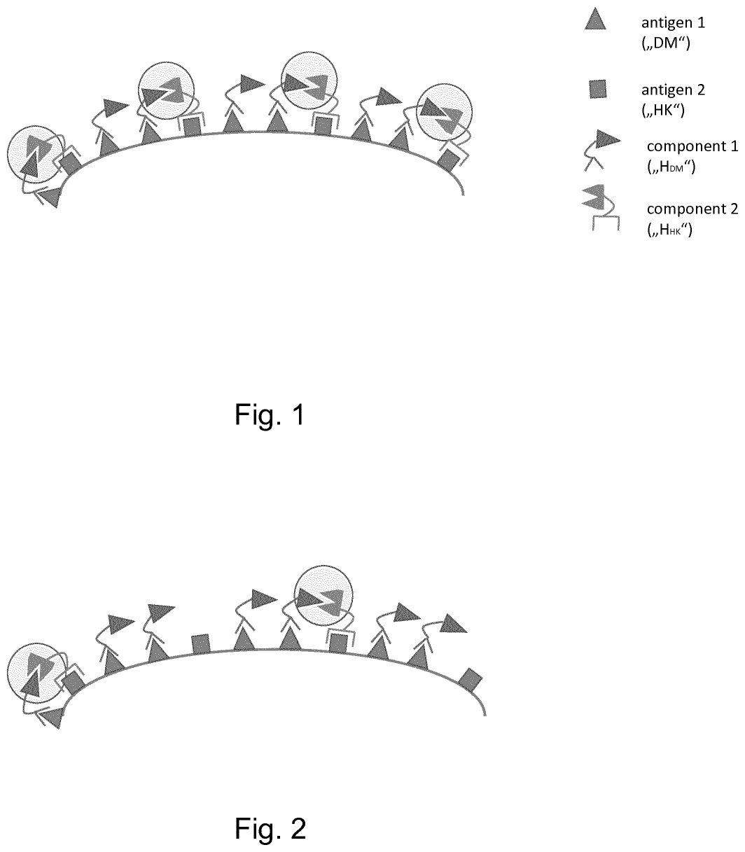

As a consequence, at least some complimentary hemibodies will associate and reconstitute a functional domain F, because the two antigens are expressed in sufficient quantity. Hence, a sufficient number of complimentary hemibodies bind to the cell surface close enough to one another so that the fragments of the functional domain of each of them can pair, or associate, to form a sufficient number of functional domains to elicit the desired function. See further explanations in the text.

FIG. 2 shows the same pathological situation with high expression of a disease marker DM (antigen 1) and physiological expression of the housekeeper HK (antigen 2). Hemibodies specific for the disease marker DM (H_{HK}) are given in high concentration so that many of them bind to the target cell. In contrast, hemibodies directed against the housekeeper antigen A_{HK} are given at non-saturating doses so that only a fraction of the housekeeper antigen is bound by the H_{HK} hemibody. Because of the high numbers of DM and consequently the high numbers of bound H_{DM} in the pathological situation of e.g. cancer, the likelihood is high that the few H_{HK} bound to the cell will find a H_{DM} hemibody partner to pair with and to reconstitute a functional domain F in sufficient numbers.

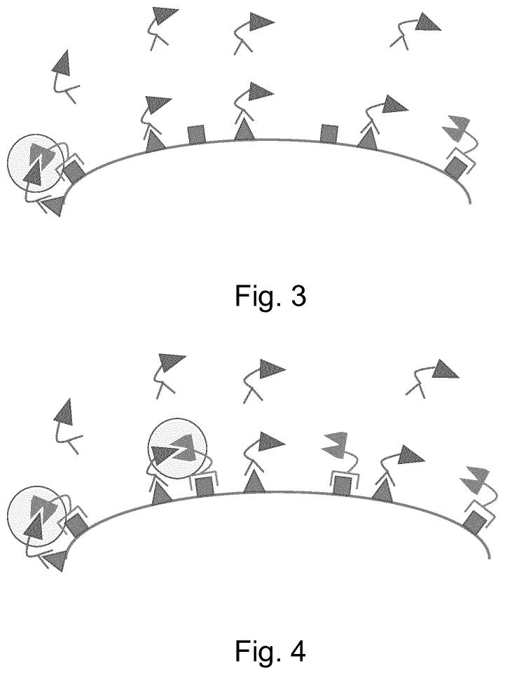

FIG. 3: In physiological situations where the disease marker DM is expressed at normal levels, only a limited number of H_{DM} can bind to the target antigen, even if applied at very high concentrations. When hemibodies directed against the housekeeping antigen A_{HK} is given at low concentrations and thus only few H_{HK} bind to the target cell, the likelihood of hemibody pairing is low. Thus, the number of reconstituted functional domains F is insufficient to trigger the desired functions.

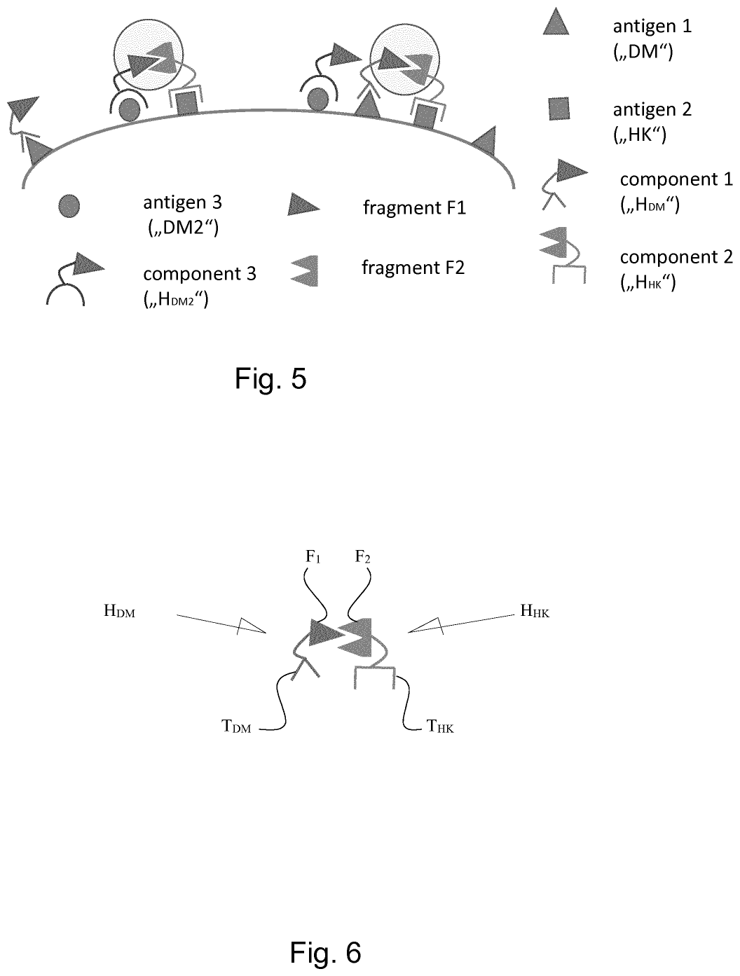

FIG. 4: In cases where the disease marker is expressed in physiological numbers, a limited number of H_{DM} will bind to the normal cell. If the housekeeping binding hemibody H_{HK} is dosed too high, a high number of H_{HK} will bind to the same normal tissue and the likelihood for reconstituting functional domains on non-target tissues increases, resulting in unintended on-target/off-tumor effects,

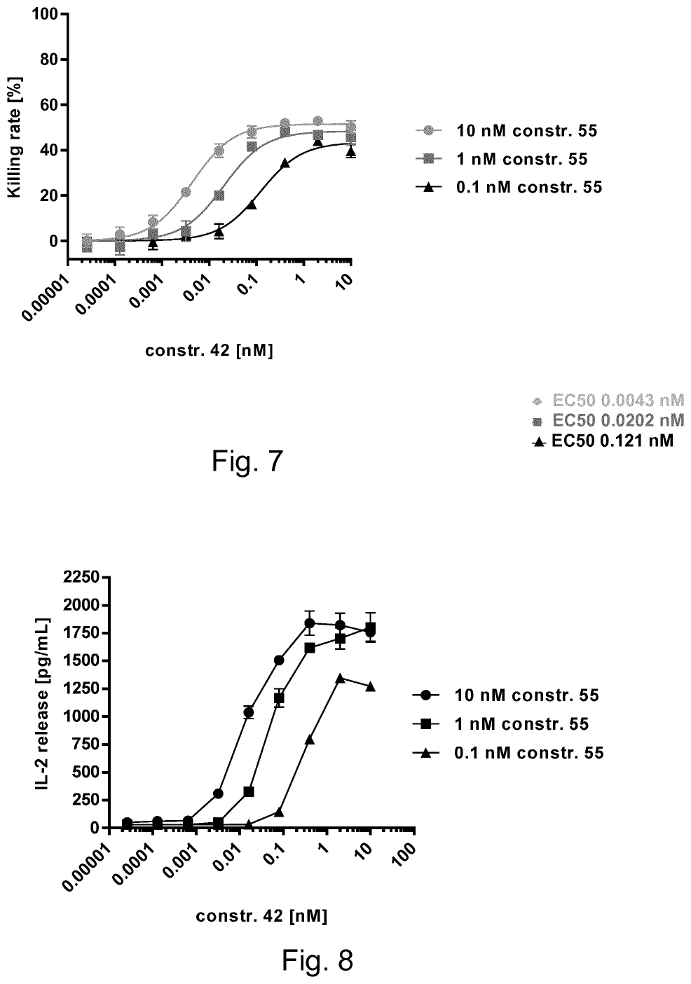

The following table summarizes these relationships:

| | | | | | | Sufficient |
|---|---|---|---|---|---|---|
| | | | | | | hemibody |
| | | | | | | pairing |
| | | | | dosing | dosing | to trigger |
| FIG. | tissue | C_{ADM} | C_{AHK} | H_{DM} | H_{HK} | function? |
| 1 | pathological | high | normal | high | high | yes |
| 2 | pathological | high | normal | high | low | yes |
| 3 | normal | normal | normal | high | low | no |
| 4 | normal | normal | normal | high | high | yes |

C_{ADM} = surface density/expression rate of disease marker
C_{AHK} = surface density/expression rate of housekeeper
H_{DM} = hemibody that binds to disease marker
H_{HK} = hemibody that binds to housekeeper

FIG. 5: In cases where two or more disease markers (DM₁, DM₂ . . . ) are expressed by the target cell, different hemibodies (H_{DM1}, H_{DM2}) can be deployed which are all equipped with the same functional fragment but differ in their targeting moiety. This way, a high number of H_{DM} hemibodies bind antigens associated with the disease and increase the likelihood that hemibody pairs H_{HK}/H_{DM1} and H_{HK}/H_{DM2} are established to reconstitute the functional domain F.

FIG. 6 shows exemplarily, a set of two complimentary hemibodies H_{HK} and H_{DM} with each hemibody comprising a different targeting moiety T_{HK} and T_{DM} which binds to a different cell surface antigen HK and DM. Further, each hemibody comprises a fragment F₁, F₂ of a functional domain. When the complimentary hemibodies bind to the cell surface close enough to one another the fragments of the functional domain of each of them can pair, or associate, to form a functional domain. See further explanations in the text.

FIG. 7: Results of the Cytotoxicity-Assay. To mimic different antigen densities on target cells, a hemibody H_{HK} specific for the surrogate housekeeper CD45 (CD3VH-scFvCD45, constr. 55) was provided at 10, 1 and 0.1 nM. A hemibody H_{DM} specific for the surrogate disease marker HLA-A2 (CD3VL-scFvHLA-A2, constr. 42) was titrated against the former.

As shown in FIG. 7, very low concentrations of H_{DM} are required for EC₅₀ (0.0043 nM) if the corresponding hemibody H_{HK} hemibody is abundantly bound to the cell (e.g., at high concentrations of 10 nM). In cases of low H_{HK} binding (at 0.1 nM, corresponding to low antigen expression at the target cell), much higher concentrations of CD3VL-scFvHLA-A2 (˜2 logs) are required to reach EC₅₀ (see FIG. 7A). This was found for induction of cytolytic activity and higher thresholds for induction of IL-2 release are needed (FIG. 8).

### DETAILED DESCRIPTION OF THE INVENTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

The inventors have surprisingly realized that the specific mode of action of hemibodies, where pharmaceutic efficacy only occurs when two or more complimentary hemibodies bind to the cell surface close enough to one another so that the fragments F1 and F2 of each of them can pair, or associate, to form a functional domain F, can be used to provide a disease specific therapy even for such pathological conditions which are not characterized by a unique and exclusive surface antigen combination, but which still feature a specific quantitative combination of densities of two or more surface antigens.

The inventors have further realized that pathological tissues exist which are not characterized by exclusive combinations of surface antigens, but which have exclusive or specific profiles regarding the quantitative absolute and ratios of copy numbers of two or more surface antigens.

For example, while Her-2/neu is expressed in healthy tissue only in small copy numbers, it is overexpressed in particular types of cancer. In contrast thereto, EpCAM is constitutively expressed in almost all epithelial cells of the human body. Hence, specific types of cancer are characterized by basal or close to basal copy numbers of a housekeeper EpCAM and high copy numbers of Her-2/neu on their cell surfaces—thus bearing a specific cell surface density ratio of Her-2/neu and EpCAM.

The inventors realized that the specific mode of action of hemibodies—where pharmaceutic efficacy only occurs when two or more complimentary hemibodies bind to the cell surface close enough to one another so that the fragments of the functional domain can pair, or associate, to form a functional domain—can be used to provide a disease specific therapy even for such pathological conditions which are not characterized by a unique and exclusive surface antigen combination, but which still feature a specific quantitative combination of densities of two or more surface antigens.

In such conditions, the application of a combination of hemibodies in a given quantity spares healthy tissue from being affected, because the binding of individual, inert hemibodies to these tissues will not cause any effect.

Thus, the inventors suggest to provide high doses of the H_{DM} hemibody directed against a disease marker which is highly expressed on the diseased cell. In contrast, the H_{HK} hemibody addressing the housekeeping antigen A_{HK} should be administered in a non-saturating dose in order to control and to titrate the formation of complementary fragments, which reach critical numbers of functional domains exclusively on cells with high density ADM expression.

In such way, patients will not be treated with the highest doses of the different hemibodies, but the hemibodies are being dosed differentially according to the expression of their different target antigens in healthy and pathological tissues. Thus, for example, the threshold for T cell activation—in case the functional domain serves to engage T-cells—can be precisely tuned by choosing an appropriate partner molecule with a desired amount of surface antigens.

According to a first aspect of the present invention, a composition comprising at least two complimentary hemibodies, is provided, wherein

the first hemibody (“H_{HK}”) comprises
(i) a fragment F₁ of a functional domain F and
(ii) a targeting moiety which binds to a cell surface antigen A_{HK} which is expressed under normal and pathological conditions (“housekeeper, HK”), and
the second hemibody (“H_{DM}”) comprises
(i) a fragment F₂ of a functional domain F and
(ii) a targeting moiety which binds to a cell surface antigen A_{DM} which is indicative for a given pathological condition (“disease marker, DM”),
and wherein the quantitative ratio H_{DM}:H_{HK} in the composition is adjusted so that, after administration to a patient,
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1,
b) the concentration ratio or the resulting serum concentration ratio H_{DM}:H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM} (pathologic tissue):C_{ADM} (non pathologic tissue) of the abundance or density of the antigen A_{DM} in a sample of cells or tissue that is a) considered, or suspected, to have, or suffer from, the pathologic condition, and b) considered healthy.

According to a second aspect of the present invention, a kit comprising at least two compositions each comprising at least one hemibody is provided, wherein

a first hemibody (“H_{HK}”) in the first composition which comprises (i) a fragment F₁ of a functional domain F and (ii) a targeting moiety which binds to a cell surface antigen A_{HK} which is expressed under normal and pathological conditions (“housekeeper”), and
a second hemibody (“H_{DM}”) in the second composition which comprises (i) a fragment F₂ of a functional domain F and (ii) a targeting moiety which binds to a cell surface antigen A_{DM} which is indicative for a given pathological conditions (“disease marker”),
and wherein, in the kit, the quantitative ratio H_{DM}:H_{HK} between the first hemibody and the second hemibody in the at least two compositions is adjusted so that, after administration to a patient,
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1,
b) the concentration ratio or the resulting serum concentration ratio H_{DM}:H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM} (pathologic tissue):C_{ADM} (non pathologic tissue) of the abundance or density of the antigen A_{DM} in a sample of cells or tissue that is a) considered, or suspected, to have, or suffer from, the pathologic condition, and b) considered healthy.

According to a third aspect of the present invention, a dosage scheme of at least two pharmaceutical compositions is provided,

wherein a combined dosage unit comprises the two pharmaceutical compositions administered to a patient simultaneously, in one unit or more units forming the combined unit, or one after the other in two or more units forming the combined unit,
wherein each pharmaceutical composition comprises one of two complimentary hemibodies, respectively,
wherein the first pharmaceutical composition comprises a first hemibody (“H_{HK}”) which comprises
(i) a fragment F₁ of a functional domain F and
(ii) a targeting moiety which binds to a cell surface antigen A_{HK} which is expressed under normal and pathologic conditions (“housekeeper”), and
wherein the second pharmaceutical composition comprises a second hemibody (“H_{DM}”) which comprises
(i) a fragment F₂ of a functional domain F and
(ii) a targeting moiety which binds to a cell surface antigen A_{DM} which is indicative for a given pathologic conditions (“disease marker”),
wherein the at least two pharmaceutical compositions are dosed in such way that, for the combined dosage unit,
the quantitative ratio H_{HK}:H_{DM} between the first hemibody and the second hemibody is adjusted so that
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1, or
b) the concentration ratio or the resulting serum concentration ratio H_{DM}:H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM (pathologic tissue)}:C_{ADM} (non pathologic tissue) of the abundance or density of the antigen A_{DM} in a sample of cells or tissue that is a) considered, or suspected, to have, or suffer from, the pathologic condition, and b) considered healthy.

As used herein, the term “housekeeper” refers to a surface antigen, which is expressed under normal and pathological conditions at a basal or near to basal rate.

As used herein, the term “disease marker” refers to a surface antigen which is overexpressed under one or more pathological conditions, but lowly expressed or not expressed under normal conditions (i.e., in a healthy tissue).

The mere presence or the expression rate of a disease marker is hence indicative for a given pathologic condition.

As used herein, the term “within a range of one order of magnitude above or below the quantitative ratio C_{ADM}:C_{AHK}” means that, for example, if the quantitative ratio C_{ADM}:C_{AHK} is 100:1, the quantitative ratio H_{DM}:H_{HK} can be in the range of between ≤1000:1 and >10:1. If, for example, the quantitative ratio C_{ADM}:C_{AHK} is 5000:1, the quantitative ratio H_{DM}:H_{HK} can be in the range of between ≤50000:1 and >500:1.

As used herein, the term “order of magnitude”, means factor 10.

The abundance or density of antigens in a sample of cells or tissue can be determined in different ways.

As used herein, the term “hemibody” relates to a set of polypeptides comprising at least
a) a first polypeptide (“hemibody H₁”) comprising (i) a targeting moiety which binds to a first antigen A₁, and (ii) a fragment F₁ of a functional domain F, and
b) a second polypeptide (“hemibody” H₂”) comprising (i) a targeting moiety which binds to a second antigen A₂, and (ii) a fragment F₂ of said functional domain F,

Notably, (i) antigen A₁ is different from antigen A₂, (ii) hemibodies H₁ and H₂ are not associated with each other in the absence of a substrate that has both antigens A₁ and A₂ at its surface, and (iii) neither fragment F₁ or F₂ alone nor hemibody H₁ or H₂ alone is functional with respect to the function of said functional domain F.

Upon dimerization of fragment F₁ of hemibody H₁ with fragment F₂ of hemibody H₂, the resulting dimer forms said functional domain F, or is functional with respect to the function of said functional domain F.

In case both hemibodies are brought in contact with a cell carrying both antigens A₁ and A₂ at its cell surface, the two hemibodies bind to the cell surface via their targeting moieties, and, in case there is a sufficient density of the said antigens on the cell surface, such dimerization of fragment F₁ of hemibody H₁ with fragment F₂ of hemibody H₂ can occur.

In contrast, if a cell does not carry both antigens A₁ and A₂, or not in a sufficient density, such dimerization does not occur, either because one or neither of the two hemibodies binds to the cell, or because the two hemibodies are too distant from one another so that dimerization in sufficient quantity is impossible

In case the two antigens are expressed on the surface of a cancer cell in sufficient density, the two hemibodies can bind and their fragments F₁ and F₂ can dimerize, to form said functional domain F, or become functional with respect to the function of said functional domain F.

The functional domain can then exert a therapeutic effect, e.g., an anti cancer effect or an immune stimulatory effect, or can server as a marker or flag.

The concept of hemibodies is broadly disclosed in WO2013104804, the content of which is incorporated herein by reference.

According to one embodiment, the composition, kit, dosage scheme or method serves to improve, or has improved, disease or target tissue specificity.

As used herein, the term “improved, disease or target tissue specificity” means improved tissue specificity compared to a targeting moieties that binds only to a disease marker.

According to one embodiment, at least one of the targeting moieties which binds to a cell surface antigen is selected from the group consisting of an
antibody, or a fragment or derivative thereof retaining target binding properties,
a Fab fragment, a F(ab′)2 fragment, a Fv (variant fragment) or a scFv (single-chain variant fragment) of an antibody.
a single domain antibody, or a non-antibody scaffold like a DARPin, an Affilin, an Ubiquitin, an Affimer, an Affitin, an Alphabody, an Anticalin, an Avimer, a Fynomer, a Kunitz domain peptide, a monobody or other antigen-binding peptides, antigen-binding proteins or aptamers.

According to one further embodiment, the surface antigen A_{HK} which is expressed under normal and pathological conditions (“housekeeper”) is at least one selected from the group consisting of:
EpCAM,
CD20,
CD45,
E-cadherin,
CEA,
EMA (epithelial membrane antigen),
αvβ6 integrin,
uPAR (urokinase-type plasminogen activator receptor), and/or
PSMA.

According to one further embodiment, the surface antigen A_{DM} which is indicative for a given pathological conditions (“disease marker”) is at least one selected from the group consisting of:
Her-2/neu,
ROR1,
VEGFR,
FGFR, and/or
EGFR.

According to one further embodiment, the fragments F₁ and F₂ of comprise subdomains of a functional domain, wherein the pairing or association of the fragments renders said functional domain functional.

According to one further embodiment, said functional domain F is at least one selected from the group consisting of antigens that trigger or bind to
a T cell engaging domain
a NK cell (natural killer cell) engaging domain,
a domain engaging macrophage cells
a monocyte/dendritic cell engaging domain
a granulocyte engaging domain
a domain engaging neutrophil granulocytes, and/or
a domain engaging activated neutrophil granulocytes, monocytes and/or macrophages.

Preferably, the NK cell (natural killer cell) engaging domain specifically binds to CD1a, CD 16a or CD56.

Preferably, the domain engaging macrophage cells specifically binds to CD16a, CD32a, CD32b, CD89 or CD64.

Preferably, the monocyte engaging domain specifically binds to CD32a, CD32b, CD64 or CD89.

Preferably, the granulocyte engaging domain specifically binds to CD 16b, CD32a, CD32b, CD64, or CD89.

Preferably, the domain engaging neutrophil granulocytes specifically binds to CD89 (FcocRI).

Preferably, the domain engaging activated neutrophil granulocytes, monocytes and/or macrophages specifically binds to CD64 (FcyRI).

According to one further embodiment, said functional domain F is a T-cell engaging domain.

Preferably the T cell engaging domain, specifically binds to CD2, CD3, CD5, T cell receptor or CD28

According to one further embodiment, said functional domain specifically binds to CD3epsilon.

According to one further embodiment, said functional domain is at least one selected from the group of

a) a target binding molecule,
b) an inflammatory or anti-inflammatory agent, and/or
c) a binder binding to at least one selected from the group consisting of a radioactive compound, or a toxic entity.

The inflammatory agent is capable of initiating an inflammatory response and to regulate the host defense against, e.g., a tumor, mediating the innate immune response.

The inflammatory agent is capable of alleviating an inflammatory response, e.g., in a tissue suffering from an autoimmune response.

A binder binding to at least one selected from the group consisting of a radioactive compound, or a toxic entity can hence accumulate toxic entities or radioactive entities in the site of disease, e.g., a tumor, when such toxic entities or radioactive entities are administered to the patient individually.

According to one further embodiment,
a) fragment F₁ comprises a VL domain of an antibody and fragment F₂ comprises a VH domain of the same antibody; or fragment F₁ comprises a V_{H} domain of an antibody and fragment F₂ comprises a V_{L} domain of the same antibody,
b) fragment F₁ comprises an antibody light chain or fraction thereof retaining target binding properties, and fragment F₂ comprises heavy chain or fraction thereof from the same antibody and retaining target binding properties; or fragment F₁ comprises an antibody heavy chain or fraction thereof retaining target binding properties, and fragment F₂ comprises a light chain or fraction thereof from the same antibody and retaining target binding properties;
c) fragment F₁ comprises a first fragment or subdomain of a target binding molecule and fragment F₂ comprises a second fragment or subdomain of the same target binding molecule.

In other embodiments, the target binding molecule can be, e.g., a non-antibody scaffold or an antibody mimetic.

In general, the two or F₁ and F₂ that are comprised in the target binding molecule can be any protein with engineered one or more CDR loops which, when associating with the respective complimentary fragment, form the functional domain as discussed herein.

Inflammatory agents in the meaning of the above definition are, for example, inflammatory cytokines like interleukin-1 (IL-1), IL-2, IL-12, and IL-18, tumor necrosis factor (TNF), interferon gamma (IFN-gamma), or granulocyte-macrophage colony stimulating factor.

Anti-Inflammatory agents in the meaning of the above definition are, for example, IL-1, IL-10, or IL-11.

In one embodiment, said functional domain specifically binding to CD3 comprises a VH domain and a VL domain selected from the group consisting of:

(i) a V domain of an anti-CD3 antibody comprising a VL domain comprising SEQ ID NOs: 18-20 (CDRs 1-3) and/or a VH domain comprising SEQ ID NOs: 15-17 (CDRs 1-3);
(ii) a V domain of an anti-CD3 antibody comprising a VL domain comprising SEQ ID NOs: 24-26 (CDRs 1-3) and/or a VH domain comprising SEQ ID NOs: 21-23 (CDRs 1-3);
(iii) a V domain of an anti-CD3 antibody comprising a VL domain comprising SEQ ID NOs: 30-32 (CDRs 1-3) and/or a VH domain comprising SEQ ID NOs: 27-29 (CDRs 1-3);
(iv) a V domain of an anti-CD3 antibody comprising a VL domain comprising SEQ ID NOs: 36 and 37 (CDRs 1 and 3) and DTS (CDR 2) and/or a VH domain comprising SEQ ID NOs: 33-35 (CDRs 1-3);
(v) a V domain of an anti-CD3 antibody comprising a VL domain comprising SEQ ID NOs: 41 and 42 (CDRs 1 and 3) and YTN (CDR 2) and/or a VH domain comprising SEQ ID NOs: 38-40 (CDRs 1-3).

In one other embodiment, said functional domain specifically binding to CD3 comprises a VH domain and a VL domain selected from the group consisting of:

(i) a V domain of an anti-CD3 antibody comprising a V_{L} domain comprising SEQ ID NO: 2 and/or a VH domain comprising SEQ ID NO: 1;
(ii) a V domain of an anti-CD3 antibody comprising a VL domain comprising SEQ ID NO: 4 and/or a V_{H} domain comprising SEQ ID NO: 3;
(iii) a V domain of an anti-CD3 antibody comprising a VL domain comprising SEQ ID NO: 6 and/or a VH domain comprising SEQ ID NO: 5;
(iv) a V domain of an anti-CD3 antibody comprising a VL domain comprising SEQ ID NO: 8 and/or a V_{H} domain comprising SEQ ID NO: 7;
(v) a V domain of an anti-CD3 antibody comprising a V_{L} domain comprising SEQ ID NO: 10 and/or a VH domain comprising SEQ ID NO: 9; and
(vi) a V domain of an anti-His antibody comprising a VL domain comprising SEQ ID NO: 12 and/or a V_{H} domain comprising SEQ ID NO: 11;
(vii) a V domain of an anti-DIG antibody comprising a VL domain comprising SEQ ID NO: 14 and/or a V_{H} domain comprising SEQ ID NO: 30.

In further embodiments, two or more RNA or DNA molecules coding for the said hemibodies are provided. Technically, such RNA or DNA molecules can be used as therapeutics, where the patient's body itself produces the respective hemibodies on the basis of the sequence information provided in the administered RNA/DNA, by protein translation. This approach is described in Stadtler et al. (2017), the content of which is incorporated herein by reference.

By adapting the dosages of the RNAs or DNAs administered and coding for HDM (high concentration) and HHK, the differential dosage as disclosed herein can be achieved.

According to one other aspect of the present invention, the use of the composition, kit, or dosage scheme according to any one of the aforementioned claims (for the manufacture of a medicament) in the treatment of a human or animal subject
being diagnosed for,
suffering from or
being at risk of
   developing a neoplastic, an autoimmune or an infectious disease is provided, or for the prevention of such condition. Alternatively, a corresponding method of treatment is provided.

### EXAMPLES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′-≥3′.

### Example 1: Dose Response Titration

For the dose response titration 10,000 luciferase-green fluorescent protein (FLuc-GFP) expressing, CD45 and HLA-A2 double positive THP1 (acute myeloid leukemia, ATCC TIB-202 and DSMZ ACC-16) cells were co-incubated with 50,000 PBMC from HLA-A2 negative healthy individuals in 100 μL in advanced RPMI-1640 supplemented with 200 μM L-glutamine, 10% FBS, penicillin (200 U/mL) and streptomycin (200 μg/mL) (Thermo Fisher Scientific, USA) in a white 96-well plate (Costar®, Corning Inc., USA). After adding serially diluted hemibody constructs, cells were further incubated under standard cell culture conditions (37° C., 5% CO₂) for 20 h before IL-2 release and Luciferase activity was assessed. IL-2 release was quantified using a IL-2 specific ELISA (IL2 ELISA Kit, ABIN1446208, antikoerper-online.de, Germany) according to manufacturer's instructions. Intracellular luciferase activity was monitored to determine killing of the firefly luciferase expressing (FLuc) THP-1 tumor cells in the presence of HLA-A2 negative PBMCs and antibody constructs. To this end, D-Luciferin (Biosynth Inc., USA) was added to a final concentration of 0.5 mM and incubated at 37° C. for 20-30 min. Subsequently, light emission was quantified with the infinite M200 pro ELISA reader (Tecan Ltd., Switzerland). Total cell killing corresponds to 100%. All assay values were statistically evaluated with the GraphPad Prism6 software (Graphpad Software, Inc., USA). Results are shown in FIG. 7 (cell toxicity assay) and FIG. 8 (IL2 release Assay)

### Example 2: Surface Protein Density

Surface binding of the constructs onto native THP-1 cells was quantified with the QIFIKIT (Dako, USA) according to manufacturer's instructions. Briefly, hemibody constructs or a BiTE where incubated with the THP-1 cells at varying concentrations on ice for 1 h before unbound construct was removed by washing with PBS. Surface bound hemibody constructs were then detected with the anti-His antibody clone AD 1.1.10 (sc-53073, Santa Cruz Biotechnology, USA) as primary antibody at a concentration of 5 μL antibody per 250 000 cells in 100 μL and with a FITC tabled secondary anti-mouse antibody supplied with the QIFIKIT. The BD FACSCalibur cytometer (BD Biosciences, USA) was used for the detection of the FITC labeled antibody.

### REFERENCES

Morgan et al., Mol Ther. 2010 April; 18(4): 843-851.
Stadler. Nat Med. 2017 July; 23(7):815-817

### Sequences

The following sequences form part of the disclosure of the present application. In case there is an ambiguity between the sequences in this table and the enclosed ST25 compatible sequence listing, the sequences in this table shall be deemed to be the correct ones.

| SEQ | Qualifier | Sequence |
|---|---|---|
| 1 | Anti CD3 VH | DVQLVQSGAEVKKPGASVKVSCKASGYTFTRYTMHWVRQAPGQGLEWIGY |
| | | INPSRGYTNYADSVKGRFTITTDKSTSTAYMELSSLRSEDTATYYCARYY |
| | | DDHYCLDYWGQGTTVTVSS |
| 2 | Anti CD3 VL | DIVLTQSPATLSLSPGERATLSCRASQSVSYMNWYQQKPGKAPKRWIYDT |
| | | SKVASGVPARFSGSGSGTDYSLTINSLEAEDAATYYCQQWSSNPLTFGGG |
| | | TKVEIKGSAAA |
| 3 | Anti CD3 VH | DIKLQQSGAELARPGASVKMSCKTSGYTFTRYTMHWVKQRPGQGLEWIGY |
| | | INPSRGYTNYNQKFKDKATLTTDKSSSTAYMQLSSLTSEDSAVYYCARYY |
| | | DDHYCLDYWGQGTTLTVSS |
| 4 | Anti CD3 VL | DIQLTQSPAIMSASPGEKVTMTCRASSSVSYMNWYQQKSGTSPKRWIYDT |
| | | SKVASGVPYRFSGSGSGTSYSLTISSMEAEDAATYYCQQWSSNPLTFGAG |
| | | TKLELK |
| 5 | Anti CD3 VH | EVQLVESGGGLVQPGGSLRLSCAASGYSFTGYTMNWVRQAPGKGLEWVAL |
| | | INPYKGVSTYNQKFKDRFTISVDKSKNTAYLQMNSLRAEDTAVYYCARSG |
| | | YYGDSDWYFDVWGQGTLVTVSS |
| 6 | Anti CD3 VL | DIQMTQSPSSLSASVGDRVTITCRASQDIRNYLNWYQQKPGKAPKLLIYY |
| | | TSRLESGVPSRFSGSGSGTDYTLTISSLQPEDFATYYCQQGNTLPWTFGQ |
| | | GTKVEIKRTIKRT |
| 7 | Anti CD3 VH | DIKLQQSGAELARPGASVKMSCKTSGYTFTRYTMHWVKQRPGQGLEWIGY |
| | | INPSRGYTNYNQKFKDKATLTTDKSSSTAYMQLSSLTSEDSAVYYCARYY |
| | | DDHYCLDYWGQGTTLTVSS |
| 8 | Anti CD3 VL | DIQLTQSPAIMSASPGEKVTMTCRASSSVSYMNWYQQKSGTSPKRWIYDT |
| | | SKVASGVPYRFSGSGSGTSYSLTISSMEAEDAATYYCQQWSSNPLTFGAG |
| | | TKLELK |
| 9 | Anti CD3 VH | EVQLVESGGGLVQPGKSLKLSCEASGFTFSGYGMHWVRQAPGRGLESVAY |
| | | ITSSSINIKYADAVKGRFTVSRDNAKNLLFLQMNILKSEDTAMYYCARFD |
| | | WDKNYWGQGTMVTVSSAKT |
| 10 | Anti CD3 VL | DIQMTQSPSSLPASLGDRVTINCQASQDISNYLNWYQQKPGKAPKLLIYY |
| | | TNKLADGVPSRFSGSGSGRDSSFTISSLESEDIGSYYCQQYYNYPWTFGP |
| | | GTKLEIKRAD |
| 11 | Anti CD3 VH | QVQLQQSGPEDVKPGASVKISCKASGYTFTDYYMNWVKQSPGKGLEWIGD |
| | | INPNNGGTSYNQKFKGRATLTVDKSSSTAYMELRSLTSEDSSVYYCESQS |
| | | GAYWGQGTTVTVSA |
| 12 | Anti CD3 VL | DYKDILMTQTPSSLPVSLGDQASISCRSSQSIVHSNGNTYLEWYLQKPGQ |
| | | SPKLLIYKVSNRFSGVPDRFSGSGSGTDFTLKISRVEAEDLGVYYCFQGS |
| | | HVPFTFGSGTKLEIKR |
| 13 | Anti CD3 VH | EVQLVESGGGLVKPGGSLKLSCAVSGFTFSDYAMSWIRQTPENRLEWVAS |
| | | INIGATYAYYPDSVKGRFTISRDNAKNTLFLQMSSLGSEDTAMYYCARPG |
| | | SPYEYDKAYYSMAYWGPGTSVTVSSAKT |
| 14 | Anti CD3 VL | DVQMTQSTSSLSASLGDRVTISCRASQDIKNYLNWYQQKPGGTVKLLIYY |
| | | SSTLLSGVPSRFSGRGSGTDFSLTITNLEREDIATYFCQQSITLPPTFGG |
| | | GTKLEIKRADAAPTVSIF |
| 15 | Anti CD3 VH | GYTFTRYTMH |
| | CDR1 | |
| 16 | Anti CD3 VH | YINPSRGYTNYADSVKG |
| | CDR2 | |
| 17 | Anti CD3 VH | YYDDHYCLDY |
| | CDR3 | |
| 18 | Anti CD3 VL | RASQSVSYMN |
| | CDR1 | |
| 19 | Anti CD3 VL | DTSKVAS |
| | CDR2 | |
| 20 | Anti CD3 VL | QQWSSNPLT |
| | CDR3 | |
| 21 | Anti CD3 VH | GYTFTRYTMH |
| | CDR1 | |
| 22 | Anti CD3 VH | YINPSRGYTNYNQKFKD |
| | CDR2 | |
| 23 | Anti CD3 VH | YYDDHYCLDY |
| | CDR3 | |
| 24 | Anti CD3 VL | RASSSVSYMN |
| | CDR1 | |
| 25 | Anti CD3 VL | DTSKVAS |
| | CDR2 | |
| 26 | Anti CD3 VL | QQWSSNPLT |
| | CDR3 | |
| 27 | Anti CD3 VH | GYSFTGYTMN |
| | CDR1 | |
| 28 | Anti CD3 VH | LINPYKGVSTYNQKFKD |
| | CDR2 | |
| 29 | Anti CD3 VH | YYGDSDWYFDV |
| | CDR3 | |
| 30 | Anti CD3 VL | RASQDIRNYLN |
| | CDR1 | |
| 31 | Anti CD3 VL | YTSRLES |
| | CDR2 | |
| 32 | Anti CD3 VL | QQGNTLPWT |
| | CDR3 | |
| 33 | Anti CD3 VH | GYTFTRYT |
| | CDR1 | |
| 34 | Anti CD3 VH | INPSRGYT |
| | CDR2 | |
| 35 | Anti CD3 VH | ARYYDDHYCLDY |
| | CDR3 | |
| 36 | Anti CD3 VL | SSVSY |
| | CDR1 | |
| 37 | Anti CD3 VL | QQWSSNPLT |
| | CDR3 | |
| 38 | Anti CD3 VH | GFTFSGYG |
| | CDR1 | |
| 39 | Anti CD3 VH | ITSSSINI |
| | CDR2 | |
| 40 | Anti CD3 VH | ARFDWDKNY |
| | CDR3 | |
| 41 | Anti CD3 VL | QDISNY |
| | CDR1 | |
| 42 | Anti CD3 VL | QQYYNYPWT |
| | CDR3 | |

1. A composition comprising at least two complimentary hemibodies, wherein
the first hemibody (“H_{HK}”) comprises
(i) a fragment F₁ of a functional domain F and
(ii) a targeting moiety which binds to a cell surface antigen A_{HK} which is expressed under normal and pathological conditions (“housekeeper, HK”), and
the second hemibody (“H_{DM}”) comprises
(i) a fragment F₂ of a functional domain F and
(ii) a targeting moiety which binds to a cell surface antigen A_{DM} which is indicative for a given pathological condition (“disease marker, DM”),
and wherein the quantitative ratio H_{DM}:H_{HK} in the composition is adjusted so that, after administration to a patient,
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1,
b) the concentration ratio or the resulting serum concentration ratio H_{DM} H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM (pathologic tissue)}:C_{ADM} (non pathologic tissue) of the abundance or density of the antigen A_{DM} in a sample of cells or tissue that is a) considered, or suspected, to have, or suffer from, the pathologic condition, and b) considered healthy.

2. A kit comprising at least two compositions each comprising at least one hemibody, wherein
a first hemibody (“H_{HK}”) in the first composition which comprises (i) a fragment F₁ of a functional domain F and (ii) a targeting moiety which binds to a cell surface antigen A_{HK} which is expressed under normal and pathological conditions (“housekeeper”), and
a second hemibody (“H_{DM}”) in the second composition which comprises (i) a fragment F₂ of a functional domain F and (ii) a targeting moiety which binds to a cell surface antigen A_{DM} which is indicative for a given pathological conditions (“disease marker”),
and wherein, in the kit, the quantitative ratio H_{DM}:H_{HK} between the first hemibody and the second hemibody in the at least two compositions is adjusted so that, after administration to a patient,
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1,
b) the concentration ratio or the resulting serum concentration ratio H_{DM}:H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM (pathologic tissue)}:C_{ADM} (non pathologic tissue) of the abundance or density of the antigen A_{DM} in a sample of cells or tissue that is a) considered, or suspected, to have, or suffer from, the pathologic condition, and b) considered healthy.

3. A dosage scheme of at least two pharmaceutical compositions,
wherein a combined dosage unit comprises the two pharmaceutical compositions administered to a patient simultaneously, in one unit or more units forming the combined unit, or one after the other in two or more units forming the combined unit,
wherein each pharmaceutical composition comprises one of two complimentary hemibodies, respectively,
wherein the first pharmaceutical composition comprises a first hemibody (“H_{HK}”) which comprises
(i) a fragment F₁ of a functional domain F and
(ii) a targeting moiety which binds to a cell surface antigen A_{HK} which is expressed under normal and pathologic conditions (“housekeeper”), and
wherein the second pharmaceutical composition comprises a second hemibody (“H_{DM}”) which comprises
(i) a fragment F₂ of a functional domain F and
(ii) a targeting moiety which binds to a cell surface antigen A_{DM} which is indicative for a given pathologic conditions (“disease marker”),
wherein the at least two pharmaceutical compositions are dosed in such way that, for the combined dosage unit,
the quantitative ratio H_{HK}:H_{DM} between the first hemibody and the second hemibody is adjusted so that
a) the concentration or the resulting serum concentration of H_{DM} is higher than H_{HK}, preferably resulting in a concentration ratio or a serum concentration ratio H_{DM}:H_{HK} of ≥2:1, more preferably ≥5:1, even more preferably ≥10:1, ≥50:1, ≥100:1, even more preferably ≥500:1, and most preferably ≥1000:1,
b) the concentration ratio or the resulting serum concentration ratio H_{DM}:H_{HK} is within a range of one order of magnitude above or below the quantitative ratio C_{ADM}:C_{AHK} of the abundance or density of the two surface antigens A_{DM} and A_{HK} in a sample of cells or tissue that is considered, or suspected, to have, or suffer from, the pathologic condition, or
c) the concentration ratio or the resulting serum concentration ratio H_{DM}:H_{HK} is within a range of one order of magnitude above or below the quantitative ratio C_{ADM (pathologic tissue)}:C_{ADM} (non pathologic tissue) of the abundance or density of the antigen A_{DM} in a sample of cells or tissue that is a) considered, or suspected, to have, or suffer from, the pathologic condition, and b) considered healthy.

4. The composition of claim 1, the kit of claim 2, or the dosage scheme of claim 3, which serves to improve, or has improved, disease or target tissue specificity.

5. The composition of claim 1, the kit of claim 2, or the dosage scheme of claim 3, wherein at least one of the targeting moieties which binds to a cell surface antigen is selected from the group consisting of an
antibody, or a fragment or derivative thereof retaining target binding properties,
a Fab fragment, a F(ab′)2 fragment, a Fv (variant fragment) or a scFv (single-chain variant fragment) of an antibody.
a single domain antibody, or a non-antibody scaffold like a DARPin, an Affilin, an Ubiquitin, an Affimer, an Affitin, an Alphabody, an Anticalin, an Avimer, a Fynomer, a Kunitz domain peptide, a monobody or other antigen-binding peptides, antigen-binding proteins or aptamers.

6. The composition of claim 1, the kit of claim 2, or the dosage scheme of claim 3, wherein the surface antigen Aux which is expressed under normal and pathological conditions (“housekeeper”) is at least one selected from the group consisting of:
EpCAM,
CD20,
CD45,
E-cadherin,
CEA,
EMA (epithelial membrane antigen),
αvβ6 integrin,
uPAR (urokinase-type plasminogen activator receptor), and/or
PSMA.

7. The composition of claim 1, the kit of claim 2, or the dosage scheme of claim 3, wherein the surface antigen A_{DM} which is indicative for a given pathological conditions (“disease marker”) is at least one selected from the group consisting of:
Her-2/neu,
ROR1,
VEGFR,
FGFR, and/or
EGFR

8. The composition of claim 1, the kit of claim 2, or the dosage scheme of claim 3, wherein the fragments F₁ and F₂ comprise subdomains of a functional domain, wherein the pairing or association of the fragments renders said functional domain functional.

9. The composition of claim 1, the kit of claim 2, or the dosage scheme of claim 3, wherein said functional domain F is at least one selected from the group consisting of
an NK cell (natural killer cell) engaging domain,
a domain engaging macrophage cells
a monocyte engaging domain
a granulocyte engaging domain
a domain engaging neutrophil granulocytes, and/or
a domain engaging activated neutrophil granulocytes, monocytes and/or macrophages.

10. The composition of claim 1, the kit of claim 2, or the dosage scheme of claim 3, wherein said functional domain F is a T-cell engaging domain.

11. The composition of claim 1, the kit of claim 2, or the dosage scheme of claim 3, wherein said functional F domain specifically binds to CD3.

12. The composition of claim 1, the kit of claim 2, or the dosage scheme of claim 3, wherein said functional domain F is at least one selected from the group of
a) a target binding molecule,
b) an inflammatory or anti-inflammatory agent, and/or
c) a binder binding to at least one selected from the group consisting of a radioactive compound, or a toxic entity.

13. The composition of claim 1, the kit of claim 2, or the dosage scheme of claim 3, wherein
a) fragment F₁ comprises a VL domain of an antibody and fragment F₂ comprises a VH domain of the same antibody; or fragment F₁ comprises a V_{H} domain of an antibody and fragment F₂ comprises a V_{L} domain of the same antibody,
b) fragment F₁ comprises an antibody light chain or fraction thereof retaining target binding properties, and fragment F₂ comprises heavy chain or fraction thereof from the same antibody and retaining target binding properties; or fragment F₁ comprises an antibody heavy chain or fraction thereof retaining target binding properties, and fragment F₂ comprises a light chain or fraction thereof from the same antibody and retaining target binding properties;
c) fragment F₁ comprises a first fragment or subdomain of a target binding molecule and fragment F₂ comprises a second fragment or subdomain of the same target binding molecule.

14. (canceled)

15. A method of treating a subject being diagnosed for, suffering from, or being at risk of developing a neoplastic disease, an autoimmune disease or an infectious disease, or for the prevention of such condition comprising administering the composition of claim 1, the kit of claim or the dosage scheme of claim 3, to a patient.
